(19) United States
(12) Patent Application Publication
(10) Pub. No.: US 2006/0217754 A1
(43) Pub. Date: Sep. 28, 2006

(54) Expandable intervertebral disc dilating cannula

(76) Inventors: Frank H. Boehm JR., Utica, NY (US); Benedetta Delorenzo Melnick, Rome, NY (US)

Correspondence Address: FRANK H. BOEHM, JR., MD, 2408 GENESEE STREET, UTICA, NY, 13502, US

(21) Appl. No.: 11/086,300

(22) Filed: Mar. 23, 2005

(51) Int. Cl. A61M 29/00

(52) U.S. Cl. 606/191

(57) ABSTRACT

In previous applications, the authors have provided Methods and Devices for minimally invasive, percutaneous methods for accomplishing disc dilation, with increase of disc height, as well as accomplishment of an intervertebral fusion, and percutaneous insertion of an artificial disc prosthesis. As an extension of these applications and inventions, the authors now provide for a method of placing an expandable cannula percutaneously into the intervertebral disc space and increasing the disc height with the use of a mechanically driven device either a pneumatically-driven device, or, as an alternative embodiment, a hydraulically-driven device. Its use for general utilization in any percutaneously achieved intervertebral procedure is also contemplated. The device provided utilizes a cannula compose of two or four leaflets that may be constructed from metal, hard plastic, or any other acceptable substance. Lying between the leaflets are expandable envelopes that may be expanded by connecting them to either a pneumatically-driven or hydraulically-driven source. As the medium of choice (fluid or gas) is driven into the envelope, the leaflets expand. In the two-leaflet model, superior and inferior leaflets are present, and these will be driven in a fashion such that the disc space is expanded in a craniocaudal direction. In the four-leaflet embodiment, in addition to expansion of the disc in a craniocaudal direction, there is also expansion in a mediolateral direction. The invention provided is intended for use in the lumbar spine. The authors contemplate the development of an analogous invention in the cervical spine.

### FIELD OF THE INVENTION

The present invention relates to a minimally invasive method for performing diagnostic and therapeutic procedures on the spine. In particular, the invention relates to a method for passing an expandable cannula into an intervertebral disc of a patient, and utilizing that cannula for either diagnostic purposes or to perform other minimally invasive procedures such as percutaneous interbody fusion as well as placement of an artificial disc through minimally invasive means.

References Cited:

| | | |
|---|---|---|
| 6,837,891 | January, 2005 | Davison, et.al |
| 6,684,886 | February, 2004 | Alleyn, Neville |
| 6,666,891 | December, 2003 | Boehm, et.al |
| 6,730,126 | May, 2004 | Boehm, et.al |

### BACKGROUND OF THE INVENTION

### Review of Related Art

Although percutaneous introduction of needles into the lumbar disc for diagnostic purposes was introduced in the 1940's, methods for treatment of disease of the lumbar spine utilizing a percutaneous approach were first introduced in the early 1980's. The first of these methods involved a percutaneous approach to decompressing a contained disc herniation (One in which the offending fragment remains completely in front of, or anterior to, the posterior longitudinal ligament. This technique, introduced by Onyk, et al, provided for a passage of a probe that was composed a chamber containing a hydraulically-driven cutting tome. The tome created multiple small fragments, which were then aspirated by an irrigation/suction modality that was also a component of the probe.

This technique enjoyed considerable notoriety and success during the late 1980's and early 1990's, but ultimately, reports, which questioned its efficacy, began to appear, and now its utilization is far more limited to certain, select cases.

Other techniques for the management of lumbar disc degeneration utilizing percutaneous approaches were also described and have enjoyed some clinical success. These included laser desiccation of the disc material; percutaneously introduced endoscopic disc surgery, and various electro-thermal modalities.

In recent years, Saul and Saul introduced the Intradiscal Electro-Thermal Discoplasty, which provided for the introduction of a copper-wire thermistor into the disc space. Over a controlled period of 12-20 minutes, the leading end of the wire is electrically heated to approximately 90 degrees Celsius, establishing a thermal injury to the integrity of the nucleus pulposus. This is thought, at least theoretically, to lead to contraction of the disc material as well as a decrease in the elasticity of the disc.

As in other novel approaches, this technique has been viewed with skepticism by members of the spinal surgery community. While numerous reports demonstrating the efficacy of this procedure have indeed been published, there have also been a considerable number of reports that call this conclusion into question. At present, the exact role that IDET procedures will ultimately have in the treatment of spinal disease remains to be elucidated.

The inventors herein, Boehm, et al, have recently proposed a system of sequential expansion of the disc space through the introduction of a system of a series of graduated catheters. This is done as a preface for the introduction of an expandable intervertebral cage to accomplish a minimally-invasive fusion.

Other investigators have introduced the concept of transforaminal, minimally-invasive procedures on the disc such as transforaminal discectomy, and even transforaminal, interbody fusion (referred to as a “T-Lift procedure) performed through a limited incision. Clearly, a trend has been established which can only be anticipated to increase.

A need, therefore, exists for a system of devices and methods of use which can be inserted into the disc space and then be expanded, so that the spectrum of procedures that can be accomplished through such an approach can be extended. It is anticipated by the inventors that the invention herein provided could be introduced for use in a wide range of procedures, including percutaneous fusion techniques as well as insertion of artificial discs via a minimally-invasive route. The cannula could also be used for diagnostic purposes insofar as evaluating whether distraction of a target disc results in temporary amelioration of a patient's symptoms. The device and method herein provided are unique, novel, and nonobvious.

### SUMMARY OF THE INVENTION

It is, therefore an object of this invention to provide a device that may be inserted through an incision in the back of a patient undergoing surgery upon the spine.

It is another object of this invention to provide a mechanism by which the device to be placed in the spine of a patient undergoing surgery is a cannula that is collapsible and expandable.

It is yet another object of this invention that the cannula that is placed in the spine of a patient undergoing surgery upon the spine composed of at least two leaflets, which, upon separation of these leaflets, will provide for distraction of the superior and inferior vertebrae relating to the disc space in a cranio-caudal direction.

It is an additional object of this invention that the distraction of the superior and inferior vertebrae results in an increase in the height of the disc in a craniocaudal direction.

It is still another object of this invention to provide an alternative embodiment that is composed of four leaflets comprising the cannula to be placed in the body of a patient undergoing surgery upon the spine.

It is still, yet, an additional object of this invention that expansion of the alternative embodiment composed of four leaflets will result in an increase in the height of the disc in a craniocaudal direction as well as an expansion of a space in a mediolateral direction.

It is still an additional, further object of this invention to create an increase in the height of the disc as well as an increase in a space in both the craniocaudal and mediolateral directions so that an implant placed into the spine may be accommodated.

It is yet another object of this invention that the configuration of the cannula in both the collapsed and expended embodiments may be either round, oval, square, rectangular, or any other shape or configuration.

It is an even further object of this invention that when the alternative embodiment of the cannula is fully expanded, distraction of the disc space in a cranio-caudal and mediolateral direction will be accomplished.

It is, additionally, another object of this invention that the expansion of the leaflets is governed by an inflatable element positioned between each pair of leaflets, regardless of whether two or four leaflets comprise the cannula to be placed in the spine of a patient undergoing surgery upon the spine.

It is yet an even further additional object of this invention that the inflation and deflation of the element that are located between each pair of leaflets is controlled and performed by either pneumatic, hydraulic, or any other means which will satisfactorily accomplish the inflation and deflation thereof.

It is still, further an object of this invention that the means for inflation and deflation of the element, whether governed by pneumatic, hydraulic, or other means, is controlled by a source that is readily and easily operated by the surgeon performing the surgery upon the spine of the patient.

It is an even further object of this invention that an alternative embodiment is provided in which the separation of the leaflets is accomplished by electro-magnetic means rather than an expandable/collapsible element.

It is an even further object of this invention that the expandable cannula can be utilized for both diagnostic and therapeutic intervention.

These and other objects of this invention are accomplished by providing a cannulated device that is composed of at least two leaflets, one serving as the inferior leaflet and one serving as the superior leaflet. The nomenclature of these leaflets is derived from their relationship to intradiscal anatomy, specifically, the superior leaflet relates to, and, in the functional embodiment of the invention, elevates the vertebral body that is superior to the disc space, while the inferior leaflet depresses the vertebral body that is inferior to the disc space. The combination of these actions results in the distraction of the disc space in a cranio-caudal direction, which, in turn, will result in the restoration of height in the setting of advanced disc degeneration. Advanced disc degeneration is generally attended by loss of disc height, and this is thought to contribute to the symptom complex by compression of the nerve roots exiting through the intervertebral foramen. The action of the elevation of the disc space results in enlargement of the intervertebral foramen, and this action and its salutary effects are more completely described below.

In addition to the embodiment described above, there is also an alternative embodiment in which there are four leaflets rather than two. In this setting, the superior and inferior leaflets are subdivided into medial and lateral embodiments. This feature allows for expansion of the space that will accommodate an intervertebral implant in a mediolateral direction in addition to the craniocaudal direction.

The patient who is undergoing surgery upon the spine is positioned in the prone position. Intraoperative fluoroscopy is utilized throughout the procedure, and the target disc space is identified. An initial guide needle is passed into the disc space, and an initial dilating cannula is passed over the guide needle into the disc space. This will expand the tract from the skin through he posterior soft tissues, thus accommodating the expandable cannula. The expandable cannula, in its collapsed form, is then passed over the dilating cannula, until it is acceptable position within the disc space, relating to the superior and inferior endplates. Regardless of whether the two or four leaflet embodiment is used, the leaflets of the cannula are held together by the expandable elements. These are attached to the free edges of the leaflets comprising the cannula. The expandable cannula is then connected to the expanding medium, whether it is hydraulic or pneumatic, and using a controlled expansion of the element, the cannula is expanded. A connecting tube carries the expanding medium from a generator to a coupling apparatus at the trailing end of the cannula. A tube composed of minimally expandable or non-expandable material is located between the leaflets at the trailing end of the cannula, which then carries the expanding medium to the expandable elements. The expanding medium is controlled by a switch, valve, or other mechanism which is under the control of the operating surgeon or his designee. As it is expanded, the disc space height is restored and the disc space is prepared for a spinal implant. This may also have diagnostic implications, if performed using local anesthesia. The restoration of the disc space height may be positively correlated with relief of the patient's symptoms, particularly radicular symptoms relating to nerve roots exiting through the foramen correlating to the disc space level. Furthermore, it can also be postulated that if the posterior longitudinal ligament is not interrupted, any disc material which has insinuated itself through the posterior annulus fibrosis may be encouraged back into the intervertebral space.

In an alternative embodiment, the mechanism governing the expansion of the cannula is neither pneumatic, or hydraulic, or any other mechanical means, but rather based on electromagnetic forces. Bearing in mind that similarly charged magnetic forces strongly repel each other, it can be recognized how that fact may be taken full advantage of.

In this embodiment, the superior and inferior leaflets have a perimeter tract located at the medial and lateral free edges, implying that in the collapsed position, these tracts closely approximate each other. Furthermore, these tracts are designed and constructed of an appropriate ferromagnetic substance such that when an electric current is input, the tracts become magnetized with either a positive or negative charge.

Given the aforementioned embodiment that is organized such that the tracts become magnetized, in order for this embodiment to be successful, further conditions must exist. Each leaflet would, logically, have a positive pole in the embodiment of one tract (medial or lateral) and a negative pole in the embodiment of the counterpart tract (medial or lateral). Therefore, if the magnetically charged tracts are arranged in such a fashion that the like charged tracts, either positive or negative, closely approximate each other, then when the tracts are magnetized (by the introduction of the electrical charge) the similarly charged tracts will repel each other, leading to the spreading of the cannula and hence the distraction of the disc space. A functional example of this would consider the case of the lateral perimeter tracts of the superior and inferior leaflets are both positively charged, with the medial tracts in both instances being [logically] negatively charged. With the introduction of the electrical charge, the positive and negative tracts, being in close approximation, will repel each other. The position of the charged tracts may be reversed, with the outcome being the same. A modulator switch would be controlled by the surgeon such that a slow, safe and controlled distraction is achieved.

Again, if this is performed on the awake, sedated patient, with good relief of pain, then one could argue that further procedures such as the performance of a fusion, or insertion of an artificial disc prosthesis, may be beneficial as well.

Obviously, in the setting of the four-leaflet embodiment, the matched “pairing” of the magnetized “perimeter tracts” is again of prima facia importance. Logic would dictate that in this setting, “like” charged tracts will be located at the supero-medial and supero-lateral tracts of the supero-lateral and supero-medial leaflets respectively. Following this paradigm, tracts carrying the same charge as the supero-medial and supero-lateral tracts above would also be found at the infero-medial and infero-lateral tracts, respectively. A charge opposite of these would then, therefore, be found at the medial and lateral pairings.

An example illustrating what is being alluded to in the paragraph above would demonstrate that in the four-leaflet model, the supero-medial leaflet, having [obviously] two free edges, would provide for a positive charge being applied to the supero-lateral edge and a negative charge being applied to the infero-medial edge. In the same example, the supero-lateral leaflet, also having two tracts, will provide for a positive charge being applied to the supero-medial edge, which is paired with the supero-lateral edge of the supero-medial leaflet. The latter is also positively charges. Hence when an electric current is applied to the device, this pair of tracts will repel each other, thus providing for expansion of the cannula in a medial-lateral direction. If the supero-lateral tract of the supero-medial leaflet is positively charged, then by definition the infero-medial tract shall be negatively charged. In a similar fashion, if the supero-medial tract of the supero-lateral leaflet is positively charged, then the infero-lateral tract of this leaflet will also be negatively charged. Therefore, these two negative charged tracts will be paired with negatively charged tracts in the infero-medial and infero-lateral leaflets respectively. Therefore, to conclude the paradigm, the infero-medial tract of the infero-lateral leaflet will be positively charged, as will the infero-lateral tract of the infero-medial leaflet. The electrical impulses which induce the magnetic charges would then be applied in a graduated fashion could be independently applied to each of the 4 pairs so that preferential craniocaudal distraction is required, more distraction is accomplished in this direction then in the medio-lateral direction.

### BRIEF DESCRIPTION OF THE DRAWINGS

FIG. 1. An elevational view of the two-leaflet embodiment of the cannula in its collapsed position.

FIG. 2. An elevational view of the two-leaflet embodiment of the cannula in its expanded position.

FIG. 3. An elevational view of the four-leaflet embodiment in its collapsed position.

FIG. 4. An elevational view of the four-leaflet embodiment in its expanded position.

FIG. 5. A view of the leading end of an alternative embodiment of the invention in which the walls of the superior and inferior leaflets of the two-leaflet embodiment are slotted, into which are fitted curved plates to prevent the expandable elements from expanding into the lumen of the cannula.

FIG. 6. A view of the leading end of an alternative embodiment of the invention in which the walls of the leaflets of the four-leaflet embodiment are slotted, into which curved plates are fitted to prevent the expandable elements from expanding into the lumen of the cannula.

FIG. 7. A lateral view of the guide needle having been placed into the target disc space under radiologic guidance.

FIG. 8. A lateral view of the non-expandable dilator having been placed over the guide needle.

FIG. 9. A lateral view of the two-leaflet embodiment of the cannula in its collapsed position, having been inserted into the target disc.

FIG. 10. A lateral view of the two-leaflet embodiment of the cannula, having been brought to the expanded position, with the vertebrae now distracted.

FIG. 11. A lateral view of the cannula, two-leaflet embodiment, connected to the mechanical source, with a regulator controlled by the operating surgeon.

FIG. 12. An anterior view of the four-leaflet embodiment in its collapsed position within the target disc space.

FIG. 13. Anterior-posterior view of the four-leaflet embodiment within the target disc space, demonstrating the increase in the disc height as well as the mediolateral space within the disc.

FIG. 14. Lateral view of the two-leaflet embodiment expanded by electro-magnetic forces.

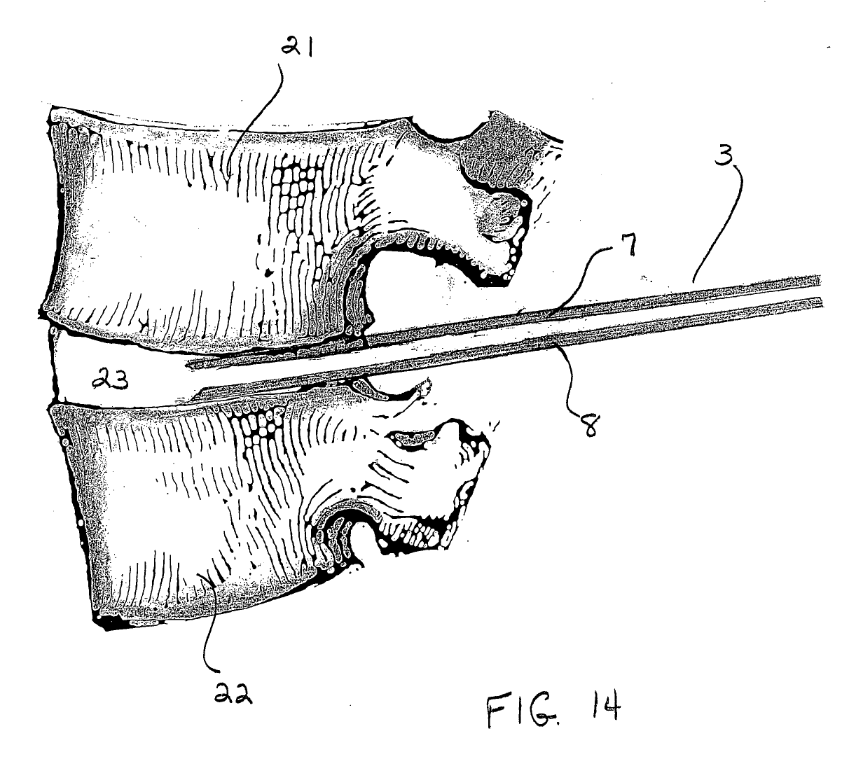

FIG. 15. Frontal view of the four-leaflet embodiment within the targeted disc space being expanded by electromagnetic means.

### DETAILED DESCRIPTION OF THE DRAWINGS

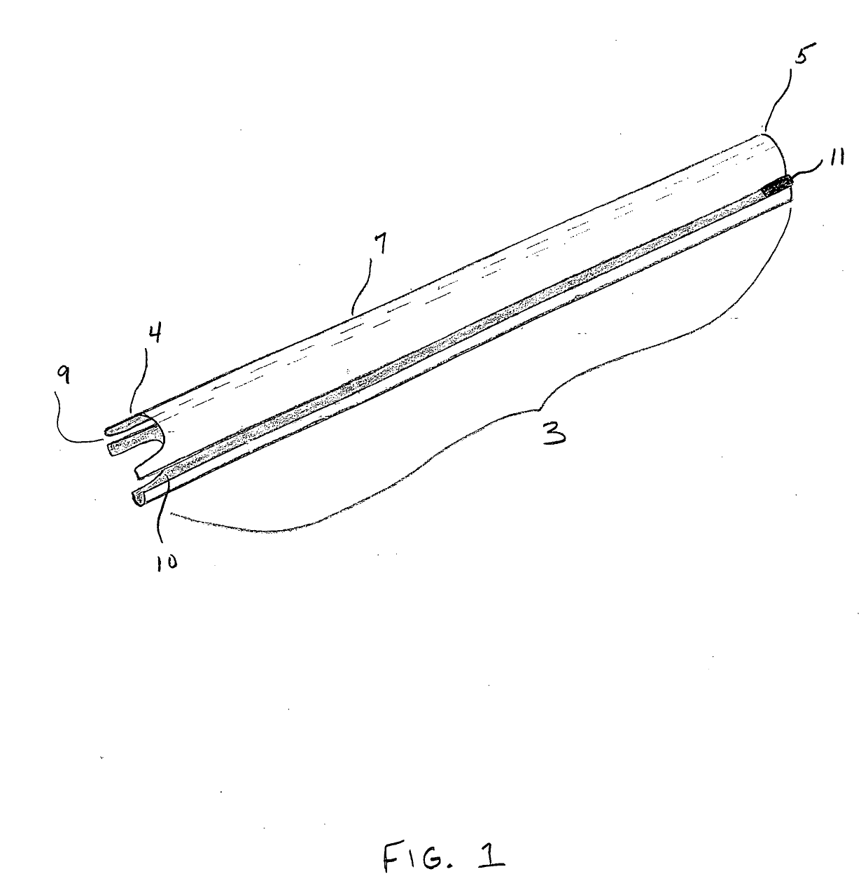

Referring now to the drawings, in which like reference numerals identify similar or identical elements throughout the many views, FIGS. 1-13 illustrate the expandable cannula of the present invention.

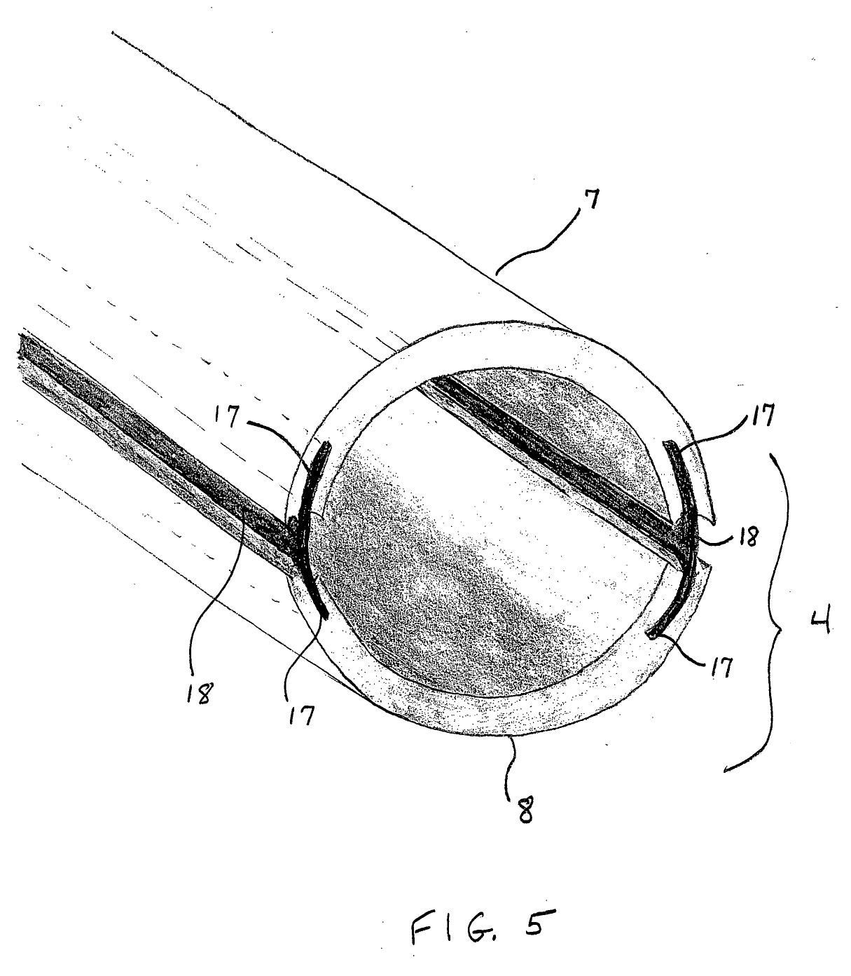

FIGS. 1-4 illustrate the various embodiments herein described and the details thereof. FIGS. 5-13 demonstrate the methods by which this invention is utilized.

Referring now to FIG. 1, which shows an elevated view of the two-leaflet embodiment 3 of the cannula containing a leading end 4 and a trailing end 5. The two-leaflet embodiment 3 is comprised of a superior leaflet 7 and an inferior leaflet 8. The expandable elements are located along the medial aspect 9 as well as the lateral aspect 10 with a connector 11 that allows the surgeon to connect to the mechanical source 12 (not shown). The connector 11 is located on the trailing end 5 of the cannula.

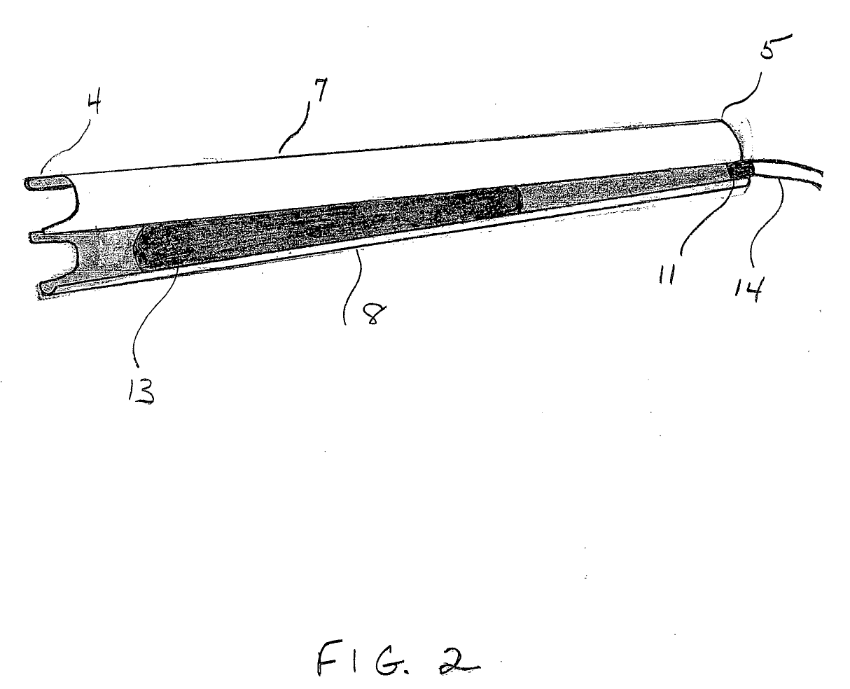

Referring now to FIG. 2 in which the expandable element 13 has been brought to its full expansion in this elevational view of the two-leaflet embodiment 3. The leading end 4 is now distracted. The trailing end 5 is connected via the connector 11 via a tube 14 to the mechanical source 12 (not shown).

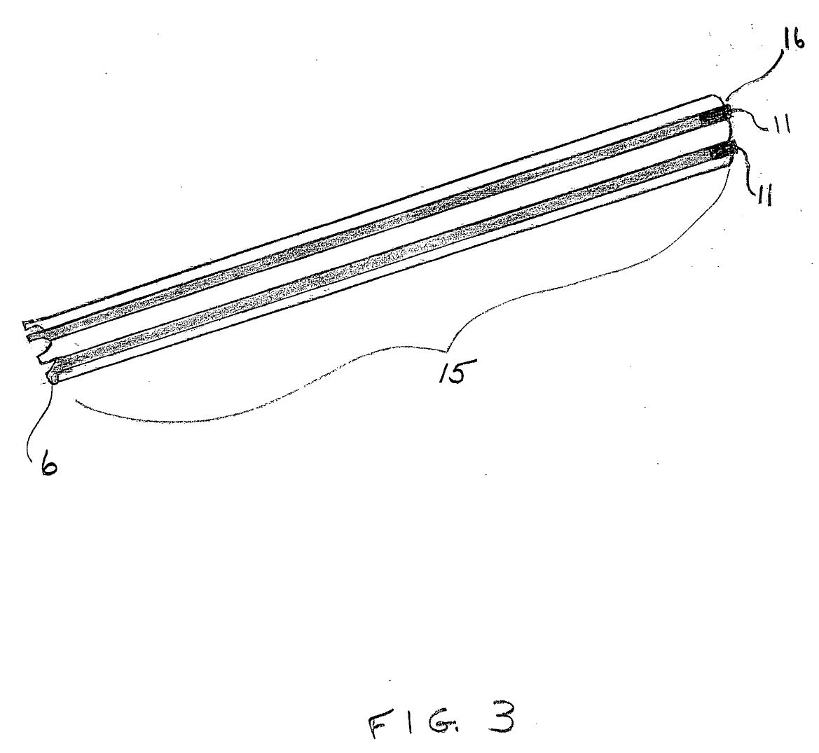

In FIG. 3 the elevational view of the four-leaflet embodiment 15 which contains a leading end 6 and a trailing end 16.

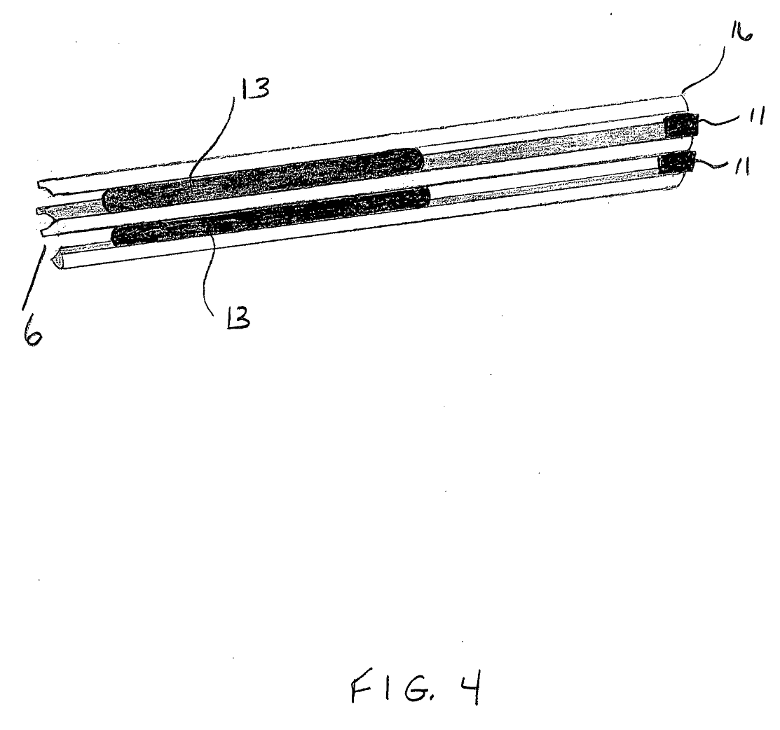

In FIG. 4 the expanded four-leaflet embodiment is seen in an elevational view. This allows for expansion of the disc space in both craniocaudal and mediolateral directions.

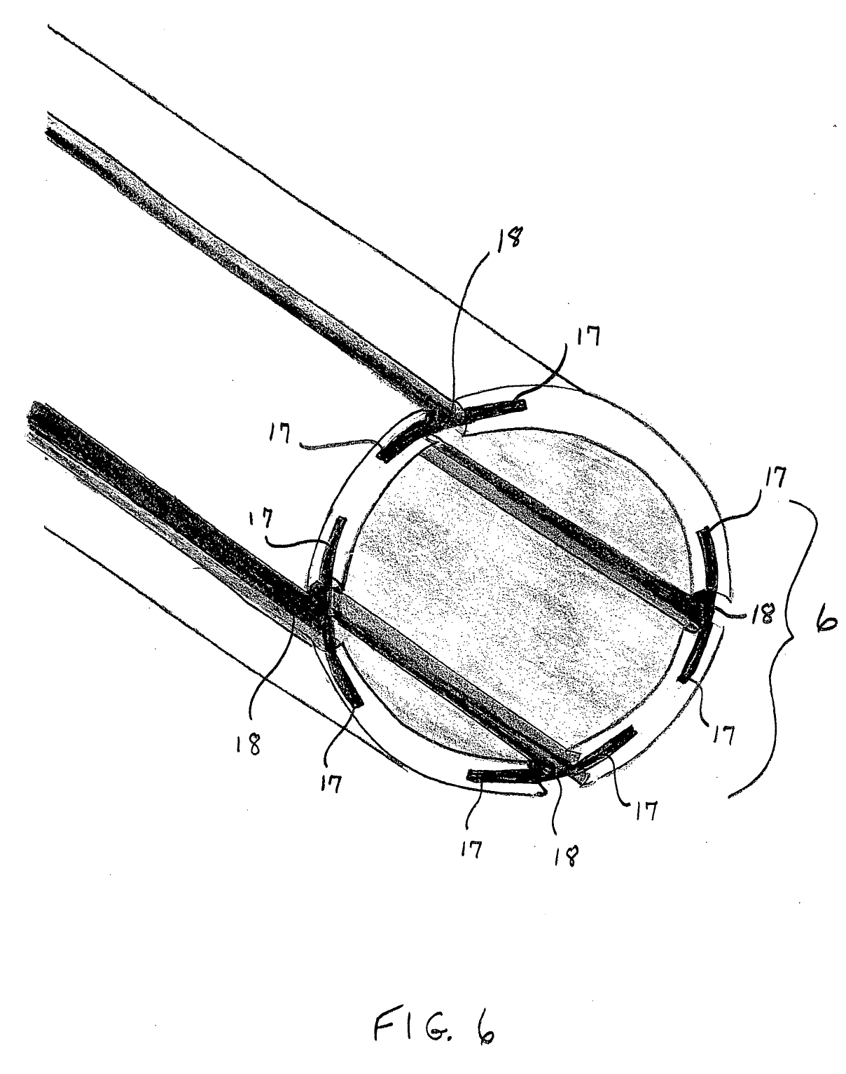

In FIGS. 5 and 6, the leading end of the two-leaflet embodiment 4 as well as the leading end of the four-leaflet embodiment 6, respectively, are demonstrated to show an alternative embodiment. In this embodiment the superior leaflet is slotted 17, as well as the inferior leaflet 17 into which plates 18 which are curved and designed to expand with the expansion of the cannula. These plates prevent the expandable elements from expanding into the lumen. Similarly in the four-leaflet embodiment, slots 17 are located on all four leaflets. Plates 18 are correspondingly positioned into each slot again to prevent the expandable element from expanding into the lumen of the cannula.

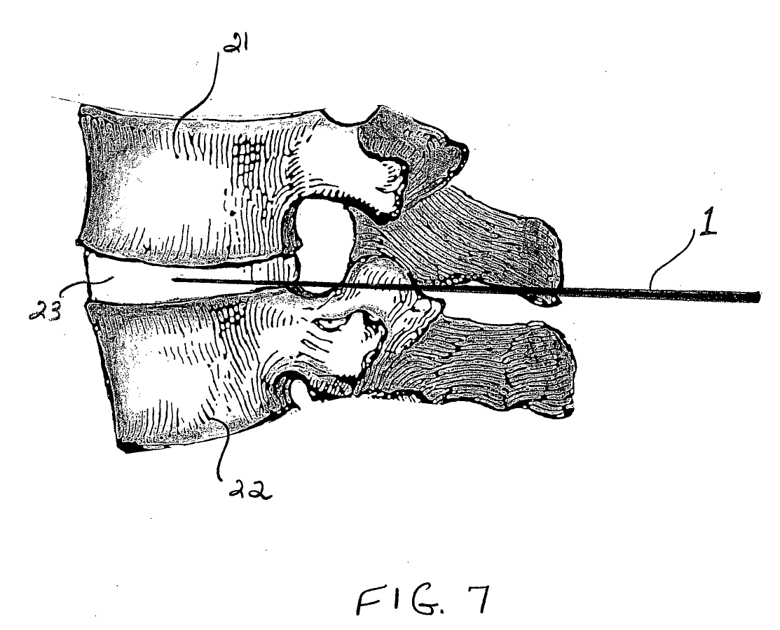

In FIG. 7 a guide needle 1 is seen on the lateral view being passed into the target intervertebral disc 23 between the superior vertebra 21 and inferior vertebra 22. This is done in the operating room under fluoroscopic guidance.

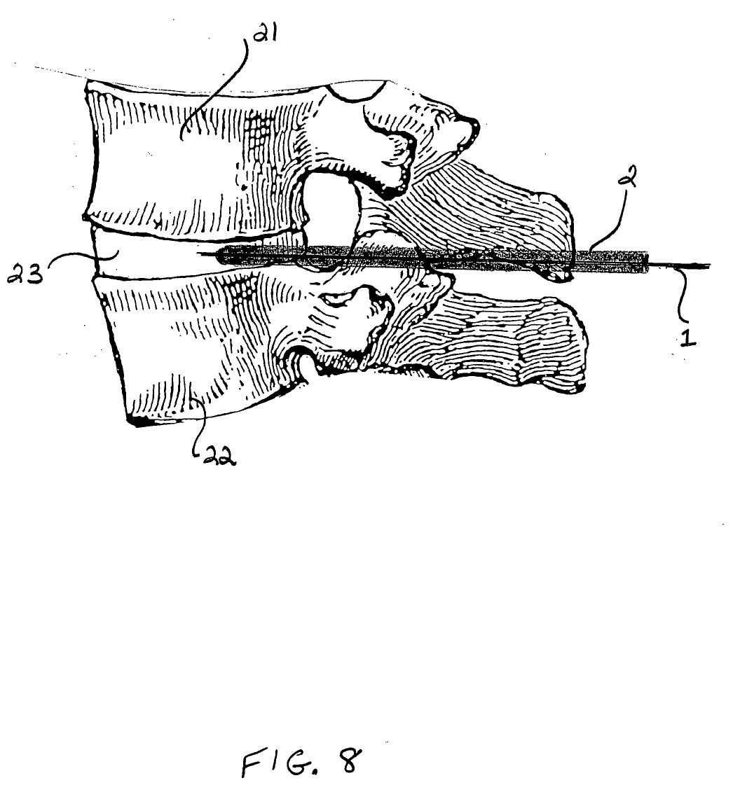

As seen in FIG. 8, a series of one or more non-expandable dilators 2 are seen being passed into the disc space 23 over the guide needle.

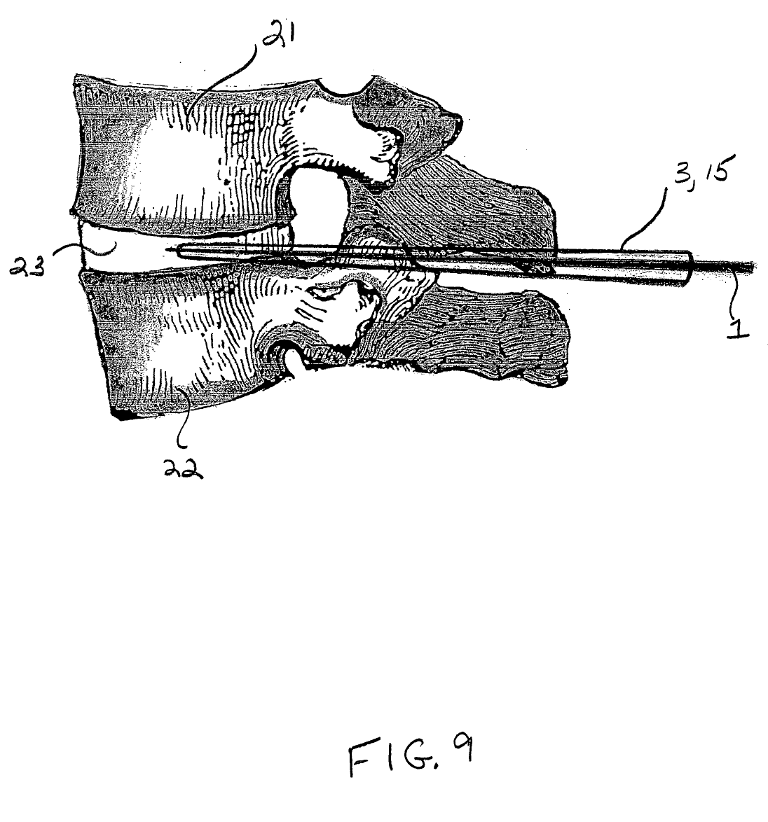

In FIG. 9, either embodiment of the expandable cannula (two-leaflet embodiment 3 or four-leaflet embodiment 15) is then passed over the guide needle 1 and non-expandable dilator 2 complex. At that point, the guide needle 1 and the non-expandable dilators 2 are removed.

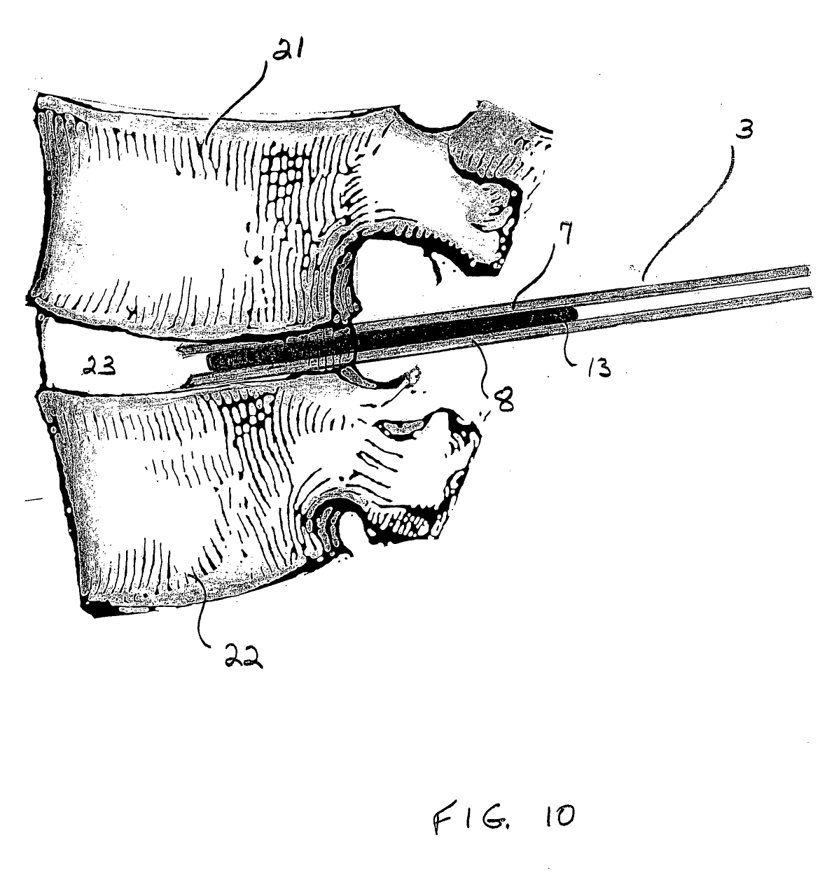

In FIG. 10, the two-leaflet embodiment of the cannula 3 has been connected to either a pneumatic or hydraulic mechanical source 12 (not shown in this figure), and the expandable elements 13 has been fully expanded. The superior leaflet 7 and the inferior leaflet 8 are now distracted, thus distracting the disc space 23. Note the posterior elements of the vertebra are not shown for illustrative purposes.

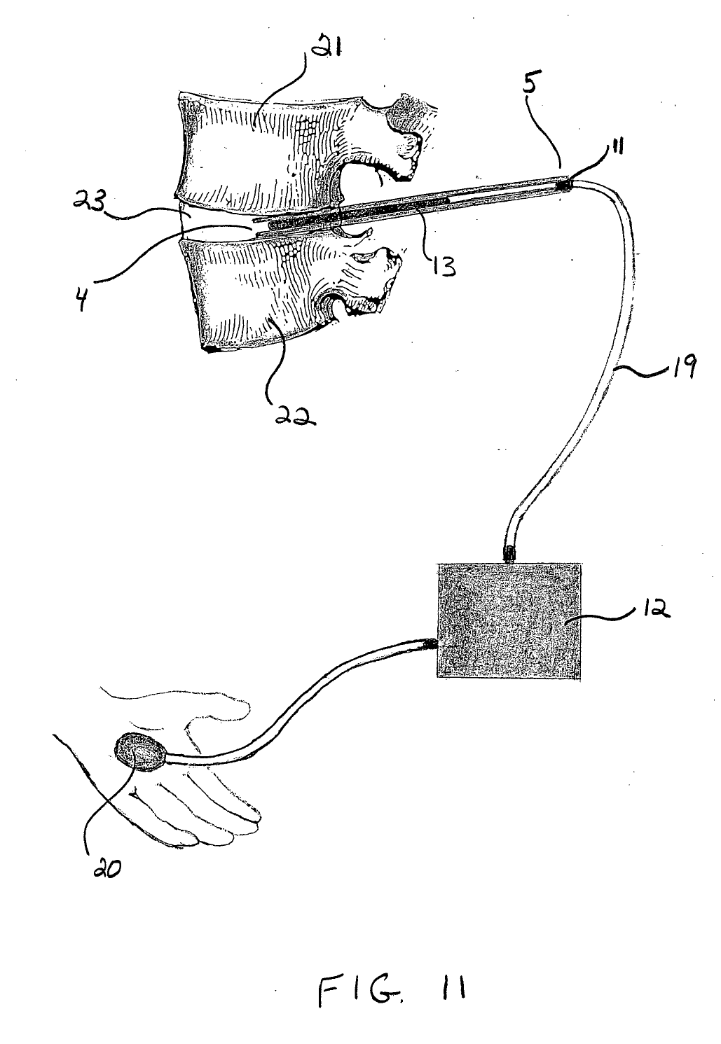

FIG. 11 demonstrates schematically the functional invention. The two-leaflet embodiment 3 has been connected by a connector 11 on its trailing end 5 to a mechanical source 12 via tubing 19. A regulator 20 is manually controlled by the surgeon allowing the leading end 4 of the cannula to distract the superior 21 and inferior 22 vertebrae. The expandable element 13 is now fully expanded, thus elevating the disc height 23. Note the posterior elements of the vertebra are not shown for illustrative purposes.

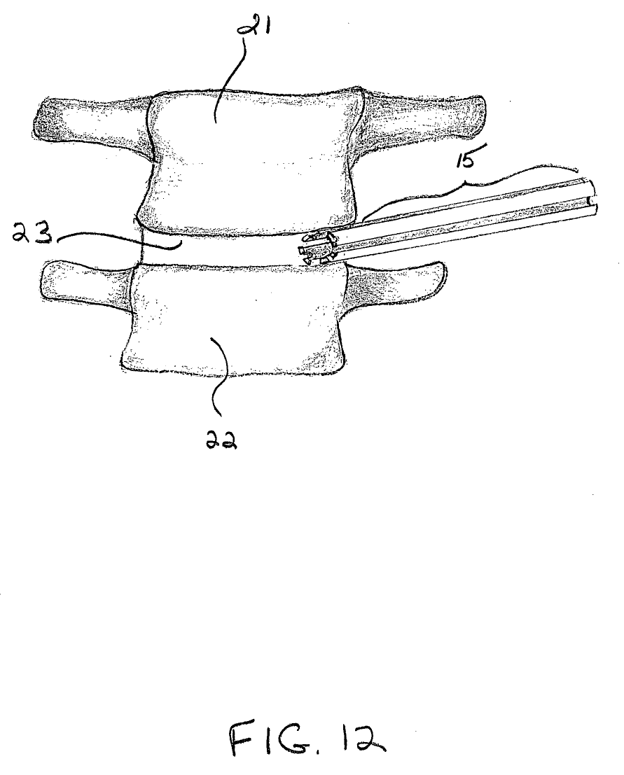

FIG. 12 shows an anterior view of the target disc 23 as well as the superior vertebra 21 and inferior vertebra 22. The four-leaflet embodiment 15 has been passed into the disc space 23 using the techniques previously described. The cannula 15 is shown in its fully collapsed embodiment in this view.

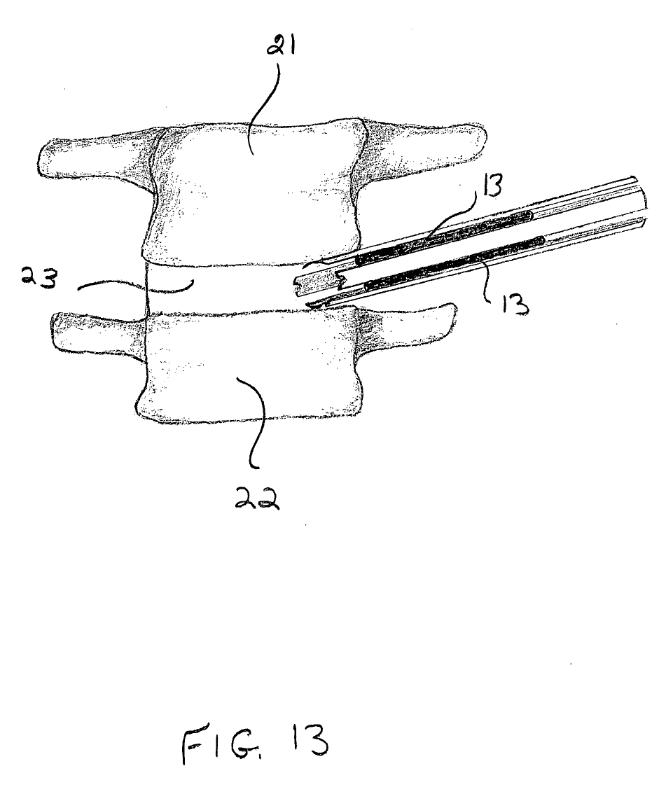

FIG. 13 shows the four-leaflet embodiment 15 in its fully expanded position with the expandable elements 13 having separated all four leaflets and in doing so, elevating the height of the disc 23 as well as creating a greater mediolateral space.

In an alternative embodiment, the two-leaflet embodiment of the cannula 3 is shown the lateral view having been positioned between the superior vertebra 21 and the inferior vertebra 22, in the intervertebral disc 23. The superior leaflet 7 and inferior leaflet 8 are distracted by means of electromagnetic force, with like charges causing these two leaflets to repel each other. Note the posterior elements of the vertebra are not shown or illustrative purposes.

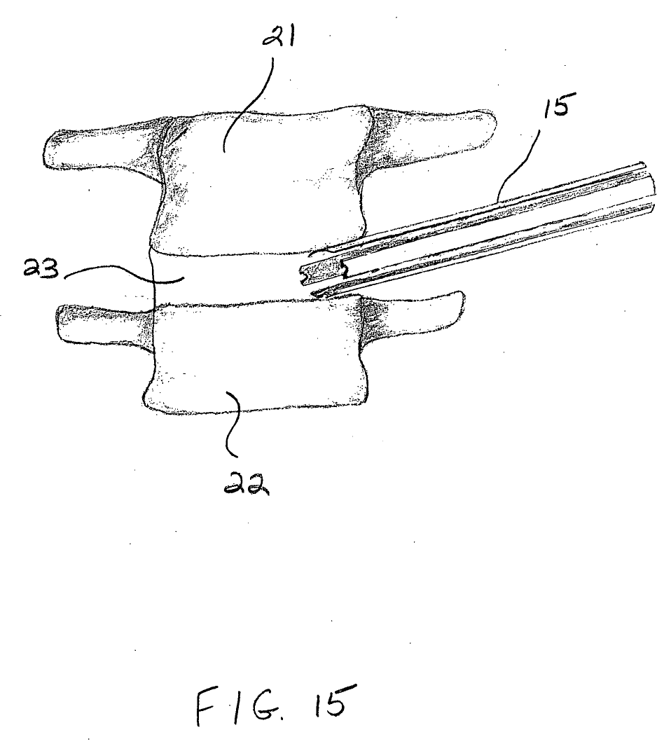

FIG. 15 shows an anterior view of the four-leaflet embodiment which has been positioned in the intervertebral disc 23. The four-leaflets have again been repelled from each other by means of electromagnetic force resulting in distraction of the disc height and a widened mediolateral space.

While the invention has been shown and described with reference to certain preferred embodiments, it will be understood by those skilled in the arts that various changes and modifications in form and detail may be made therein without departing from the spirit and scope of the invention as defined by the appended claims.

We claim:

1. An expandable cannula system, which can be inserted into the spine of the patient and specifically into the intervertebral disc, consisting of:
At least two or more leaflets, each of which has a leading and a trailing end, with expandable elements interposed between each of these said leaflets; the cannula being composed of these leaflets may be round, oval, square, or any other configuration in both the collapsed and expanded configuration;
A series of expandable elements composed of rubber, silicone, polymer, or any other expandable material that is located along and between the free edges of the leaflets at each location wherein theses leaflets appose each other;
A system of devices that will create expansion of the expandable elements, with either the use of pneumatic, hydraulic, or any other mechanical drive system;
The expansion of the expandable elements will lead to preferential expansion of the cannula at the leading end, which, depending on the number of leaflets, results in distraction in a craniocaudal direction or distraction in both a craniocaudal and mediolateral direction, but will not expand into the lumen of the cannula;
A unique feature of the leading end of the cannula such that regardless of the number of leaflets comprising a particular embodiment, the leading end of the cannula is arranged in such a fashion that the medial and lateral aspects of the cannula extend beyond the superior and inferior aspects of the cannula, thus creating a space into which an implant may be secured into the disc space.

2. A system of methods and devices for insertion and expansion of the expandable cannula in claim 1, consisting of:
A guide needle that is placed into the target disc space under fluoroscopic guidance;
A series of one or more non-expandable dilators to prepare the tract for the passage of any embodiment of the expandable cannula;
The expandable cannula in claim 1, consisting of two or more leaflets, which is passed over the outermost non-expandable dilators;
The expandable elements in claim 1, which are positioned between each two leaflets, and which will preferentially expand in such a fashion that the leading end of the cannula, regardless of the number of leaflets, will expand more than the trailing end;
A mechanical source, providing either pneumatic, hydraulic, or any other mechanical source of expansion medium to the expandable elements;
A tube or series of tubes carrying the expansion medium to the expandable elements;
A connector, or series of connectors/intact valves, connecting the tube(s) carrying the medium to the expandable elements;
A regulator allowing the surgeon to regulate the amount of expansion medium that is transmitted to the expandable elements, and thereby regulating the amount of expansion the cannula will achieve.

3. The expandable elements in claims 1 and 2 are connected to the free edges of the leaflets so that the entire complex functions as a cannula.

4. An alternative embodiment to insure that the expandable element do not expand into the cannula consisting of:
A series of slots located within the walls of the leaflets of the cannula;
A series of curvilinear plates, which are made of metal, hard plastic or any other appropriate substance, and which are found positioned within the slots of the leaflets and positioned in such a way that the expandable elements are prevented by these plates from expanding into the lumen.

5. An alternative embodiment, in which the expansion of the cannula is accomplished by electromagnetic forces.

6. In the alternative embodiment in claim 5 above, there are tracts found along the free edges of each leaflet, regardless of the number of leaflets, these tracts being composed of a metal element, compound or alloy which can be reversibly magnetized by the input of electricity or any other input.

7. In the alternative embodiment in claims 5 and 6 above, the tracts are arranged so that like-charged tracts, either positive or negative, are aligned parallel to each other, resulting in repulsion of the respective leaflets, and hence expansion of the cannula, when the tracts are magnetized.

8. In the alternative embodiment in claims 5, 6, and 7 above:
A power source, be it electrical or otherwise, to reversibly magnetize the tracts;
A means of carrying the power source to the tracts;
A regulator, by which the surgeon may control the amount of magnetization, and therefore the amount of expansion of the cannula.
